(19) United States
(12) Patent Application Publication
(10) Pub. No.: US 2007/0276154 A1
(43) Pub. Date: Nov. 29, 2007

(54) PROCESSES FOR THE PRODUCTION OF ORGANIC ISOCYANATES

(75) Inventors: Michel Haas, Dormagen (DE); Tim Loddenkemper, Dormagen (DE); Bernd Ruffer, Leverkusen (DE)

Correspondence Address: CONNOLLY BOVE LODGE & HUTZ, LLP, P O BOX 2207, WILMINGTON, DE, 19899, US

(73) Assignee: Bayer Material Science AG, Leverkusen (DE)

(21) Appl. No.: 11/752,395

(22) Filed: May 23, 2007

(30) Foreign Application Priority Data
May 23, 2006 (DE) 102006024549.0

(51) Int. Cl. C07C 263/10; C07C 51/58

(52) U.S. Cl. 560/347; 562/847

(57) ABSTRACT

Processes for the production of organic isocyanates, comprising the production of phosgene by reaction of CO with Cl₂, the reaction of the phosgene with organic amines to form the organic isocyanates, and the separation of the organic isocyanates, which is characterised in that the carbon monoxide is removed from the HCl-containing waste gas from the isocyanate synthesis by reaction with chlorine to form phosgene. The phosgene can be separated off and can optionally be fed back into an isocyanate synthesis The HCl-containing, CO-depleted gas is preferably subjected to HCl oxidation (Deacon). A closed chlorine cycle can be used in the isocyanate synthesis.

### BACKGROUND OF THE INVENTION

Many chemical processes which include a reaction with chlorine or phosgene, such as the production of isocyanates or chlorination reactions of aromatic compounds, lead to an accumulation of hydrogen chloride. Such accumulated hydrogen chloride can be converted back to chlorine by electrolysis, such as described in, for example, WO97/24320A1. In comparison to this type of energy-intensive method, the direct oxidation of hydrogen chloride with pure oxygen or an oxygen-containing gas in the presence of a heterogeneous catalyst (such as, for example, what is often referred to as the Deacon process) according to the following reaction
4HCl+O₂⇄2Cl₂+2H₂O
offers advantages in terms of energy consumption. Such a process is described, for example, in WO 04/014845, the entire contents of which are incorporated herein by reference.

In many processes which include a reaction with chlorine or phosgene, such as in particular phosgenation, a relatively large amount of carbon monoxide (CO) can be included in the resulting HCl containing waste gas as an impurity. In the generally widely used liquid phase phosgenation reactions, carbon monoxide in an amount from 0 to 3 vol. % can be found in the HCl waste gas from the phosgene scrubbing column. In state-of-the-art gaseous phase phosgenations, even higher CO amounts (up to more than 5%) can be expected, since in such methods preferably no condensation of phosgene, and therefore no associated large scale separation of the unreacted carbon monoxide, is carried out before the phosgenation.

In the conventional catalytic oxidation of hydrogen chloride with oxygen, a very wide range of catalysts can be employed, e.g., based on ruthenium, chromium, copper, etc. Such catalysts are described, for example, in DE1567788 A1, EP251731A2, EP936184A2, EP761593A1, EP711599A1 and DE10250131A1, the entire contents of each of which are herein incorporated by reference. Such catalysts can however at the same time act as oxidation catalysts for other components that may be present in a reaction stream, such as carbon monoxide or various organic compounds. The catalytic carbon monoxide oxidation to carbon dioxide is however extremely exothermic and can cause uncontrolled local temperature rises (hot spots) at the surface of heterogeneous catalysts, with the result that a deactivation of the catalyst with respect to the HCl oxidation may occur. For example, without cooling, the oxidation of 5% carbon monoxide in an inert gas (e.g., N₂) at an inflow temperature of 250° C. (described operating temperatures in Deacon processes are generally 200°-450° C.) would result in a temperature rise of far above 200° C. One likely reason for the catalyst deactivation may be microstructural change of the catalyst surface, e.g., by sintering processes, on account of the formation of hot spots.

Furthermore the adsorption of carbon monoxide on the surface of the catalyst cannot be excluded. The formation of metal carbonyls may take place reversibly or irreversibly and may thus occur in direct competition to the desired HCl oxidation. Carbon monoxide can, at high temperatures, form very stable bonds with some elements, such as, e.g., osmium, rhenium, ruthenium (see, e.g., CHEM. REV. 103, 3707-3732, 2003), and may thereby inhibit the desired target reaction. A further disadvantage could arise due to the volatility of such metal carbonyls (see, e.g., CHEM. REV. 21, 3-38, 1937), whereby not inconsiderable amounts of catalyst are lost and in addition, depending on the application, an expensive and complicated purification step of the reaction product can be necessary.

Processes for the oxidation of hydrogen chloride with oxygen in which the carbon monoxide content of the gas that is used is adjusted in advance to less than 10 vol. % by palladium-catalysed combustion to form carbon dioxide, separation of the hydrogen chloride gas by distillation, or scrubbing of the gas with a solution of copper chloride to extend the lifetime of the catalyst, have been suggested.

In another known process a hydrogen chloride-containing waste gas is fed into an aqueous alkaline absorption system and the waste gas freed from hydrogen chloride and phosgene is sent to a combustion plant.

A disadvantage of the previously suggested processes for overcoming the aforementioned problems is the destruction of the a valuable carbon monoxide raw material along with its removal.

Therefore, it would be desirable to separate the carbon monoxide from such hydrogen chloride-containing waste gases, in order to prevent disadvantages caused thereby in a subsequent Deacon process, and simultaneously make use of the carbon monoxide in an economic manner.

### BRIEF SUMMARY OF THE INVENTION

The present invention relates, in general, to processes for the production of organic isocyanates, comprising: producing phosgene by reaction of carbon monoxide (CO) with chlorine (Cl₂), reacting the phosgene with an organic amine to form an organic isocyanate and a waste gas including hydrogen chloride and carbon monoxide, and separating the organic isocyanate from the waste gas, wherein the carbon monoxide is removed from the HCl-containing waste gas by reaction with chlorine to form phosgene. The phosgene can be separated off and can optionally be fed back into an isocyanate synthesis. The HCl-containing, CO-depleted gas is preferably subjected to HCl oxidation (Deacon). A closed chlorine cycle can thus be provided in the isocyanate synthesis.

The present inventors have discovered a significant advantage in reacting the carbon monoxide found in an HCl waste gas from an isocyanate synthesis with chlorine to form phosgene, to further separating the resulting phosgene and, in feeding the phosgene back into an isocyanate synthesis or phosgenation reaction. The substantially CO-free waste gas can be fed into a Deacon process, it being possible for the resulting chlorine to be reused for the production of phosgene. Processes according to the invention alleviate the need for the separation of CO from the phosgene by the particularly energy-consuming condensation of the phosgene. Carbon monoxide can be left in the phosgene during isocyanate formation, and subsequently separated from the waste gas prior to HCl oxidation by a process according to the invention. In a Deacon process, the risk of the formation of hot spots and the associated catalyst deactivation due to the exothermic formation of CO₂ from CO can be avoided. Furthermore, no accumulation of carbon dioxide occurs in the recycling stream in the Deacon process.

One embodiment of the present invention includes a process for the production of organic isocyanates, comprising: reacting carbon monoxide with chlorine to form phosgene; reacting the phosgene with an organic amine to form an organic isocyanate and a waste gas comprising hydrogen chloride and carbon monoxide; reacting the carbon monoxide in the waste gas with chlorine to form an intermediate gas comprising additional phosgene; and separating the additional phosgene from the intermediate gas.

In a preferred embodiment of a process according to the present invention, the process further comprises supplying the additional phosgene to an isocyanate synthesis.

In another preferred embodiment of a process according to the present invention, the process farther comprises subjecting the intermediate gas to HCl oxidation.

In yet another preferred embodiment of a process according to the present invention, the process further comprises supplying the additional phosgene to an isocyanate synthesis and subjecting the intermediate gas to HCl oxidation.

Preferably, the hydrogen chloride-containing intermediate gas can be supplied to a Deacon process after separating off the additional phosgene.

Production of the phosgene and the organic isocyanate, and the separation of the isocyanate can each be performed in a manner that is known per se, and reference may be made to the relevant prior art in each respect.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there is shown in the drawing an embodiment which is presently preferred. It should be understood, however, that the invention is not limited to the precise arrangements and instrumentalities shown.

In the Figs.:

FIG. 1 is a flow chart illustrating a process according to one embodiment of the present invention;

FIG. 2 is a flow chart illustrating a conventional process which includes phosgene condensation; and

FIG. 3 is a flow chart illustrating a process in accordance with another embodiment of the present invention.

### DETAILED DESCRIPTION OF THE INVENTION

As used herein, the singular terms “a” and “the” are synonymous and used interchangeably with “one or more.” Accordingly, for example, reference to “a gas” herein or in the appended claims can refer to a single gas or more than one gas. Additionally, all numerical values, unless otherwise specifically noted, are understood to be modified by the word “about.”

In the processes according to various embodiments of the invention, a gas containing hydrogen chloride (HCl) and carbon monoxide (CO), which results from the production of isocyanate by reaction of organic amines with phosgene, is subjected to reaction with chlorine to separate the carbon monoxide (CO), by reaction with the chlorine to form phosgene. The hydrogen chloride-containing gases resulting from the isocyanate production can contain, for example, 0.1 to 20 vol. %, preferably 0.5 to 15 vol. %, carbon monoxide. The content of hydrogen chloride can be, for example, 20 to 99.5 vol. %, preferably 50 to 99.5 vol. %. The remaining gases in the hydrogen chloride-containing gas includes, e.g., nitrogen, oxygen, carbon dioxide and noble gases. These may be present, for example, in an amount of approximately 0.5 to 80 vol. %

The reaction of carbon monoxide in the hydrogen chloride-containing waste gas is carried out by the reaction of carbon monoxide with chlorine to form phosgene, for example, on an activated carbon catalyst. Alternative catalysts can also be used however. Such suitable catalysts are described, for example, in DE 3327274; GB 583477; WO 97/30932; WO 96/16898; and U.S. Pat. No. 6,713,035, the entire contents of each of which are herein incorporated by reference.

In one particularly preferred embodiment of a process according to the invention, the reaction of carbon monoxide with chlorine to form phosgene is carried out using activated carbon as a catalyst in a fixed bed reactor with a slight molar excess chlorine of (around 1.0 to 1.5 mol of Cl₂ per mol of CO), a temperature of about 20 to 600° C., and a pressure of about 1 to 20 bar.

Operating under pressure can allow the size of the reaction vessel to be reduced and can simplify the subsequent separation of the phosgene that can be carried out in various preferred embodiments.

In contrast to the conventional production of phosgene, processes according to the invention for separating carbon monoxide can be performed with a molar chlorine excess in order to separate the carbon monoxide as completely as possible. The excess chlorine does not interfere with the chlorine oxidation process that preferably follows, as it is formed in any case. Conventional phosgene production is performed with an excess of carbon monoxide to prevent residues of chlorine in the phosgene produced.

After reaction of carbon monoxide with Cl₂, the phosgene that is formed can be separated by at least one operation selected from liquefaction or condensation of the phosgene; liquefaction (with cooling and/or under pressure) can optionally take place after first drying the gas mixture, as described, for example, in DE-A-1567599 and GB 737442, the entire contents of each of which are incorporated herein by reference; (it should be emphasised here that the amount of phosgene liquefied here is naturally much smaller than the amount of phosgene that would have to be liquefied after the actual phosgene production process to separate off the CO.); distillation or rectification and/or; scrubbing of the phosgene with a solvent, such as, e.g., monochlorobenzene or ortho-dichlorobenzene.

Separation of the phosgene by condensation or distillation is preferred.

According to various embodiments of the invention, the phosgene separated off in this way is preferably returned to a phosgenation reaction, such as, for example, in an isocyanate production process. The separated phosgene is particularly preferably returned to the same phosgenation reaction in which the hydrogen chloride-containing waste gas used according to the invention was formed.

According to various embodiments of the invention, after separation of the phosgene the hydrogen chloride-containing, carbon monoxide depleted gas (i.e., the intermediate gas) preferably undergoes catalytic oxidation with oxygen in a manner known per se. The commonly accepted name for this process is the “Deacon process”. With regard to the performance of HCl oxidation, reference can be made to the relevant prior art.

The intermediate hydrogen chloride-containing, carbon monoxide depleted gas preferably has a CO content of less than 1 vol. %, more preferably less than 0.5 vol. %.

Preferred parameters for the catalytic oxidation of HCl in accordance with one embodiment of the present invention include the use of: ruthenium, chromium, copper, bismuth compounds as the catalyst; a molar ratio of HCl:O₂: of 4:1 to 1:1; a temperature of 200 to 450° C.; a pressure of 1 to 100 bar; in a fixed bed, fluidised bed, or micro-reactor; under isothermal or adiabatic conditions.

Preferred parameters for the catalytic oxidation of HCl in accordance with one embodiment of the present invention include the use of ruthenium, chromium, copper, bismuth compounds as the catalyst; a molar ratio of HCl:O₂: of 4:1 to 1:1; a temperature of 200 to 450° C.; a pressure of 1 to 100 bar; in a fixed bed, fluidised bed, or micro-reactor; under isothermal or adiabatic conditions.

Various particularly preferred embodiments of processes according to the invention comprise: (a) production of phosgene by reacting CO with Cl₂; (b) subsequent use of the thus produced phosgene in a reaction with an organic amine to form an organic isocyanate and an HCl-containing waste gas (according to various embodiments of the invention, this step can preferably be carried out without the previous separation of the CO); (c) separation of the organic isocyanate thus obtained; (d) separation of the carbon monoxide from the HCl-containing waste gas resulting from the isocyanate synthesis by reaction with chlorine to form phosgene; (e) separation of the phosgene formed; (f) recirculation of the phosgene formed into the isocyanate synthesis; (g) subjecting the HCl-containing, CO-depleted gas to HCl oxidation and recirculation of the Cl₂ formed into the production of the phosgene, which can be both the initial phosgene production and the subsequent phosgene production in the context of the separation of CO from the HCl process gas.

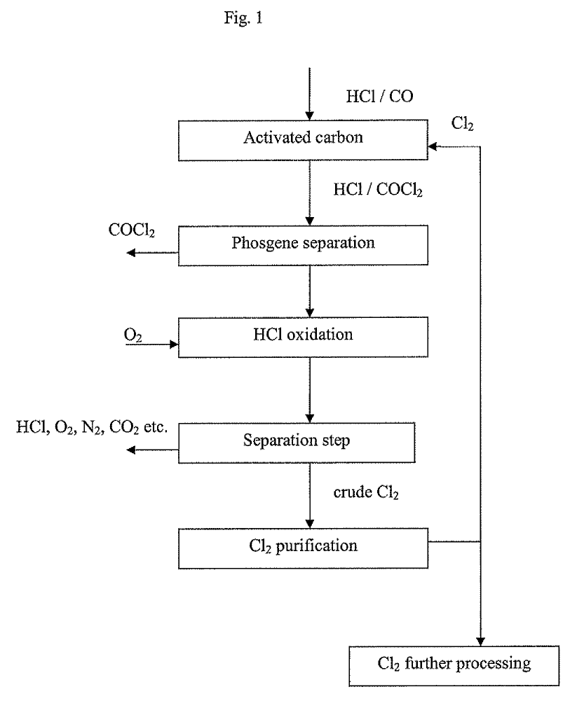

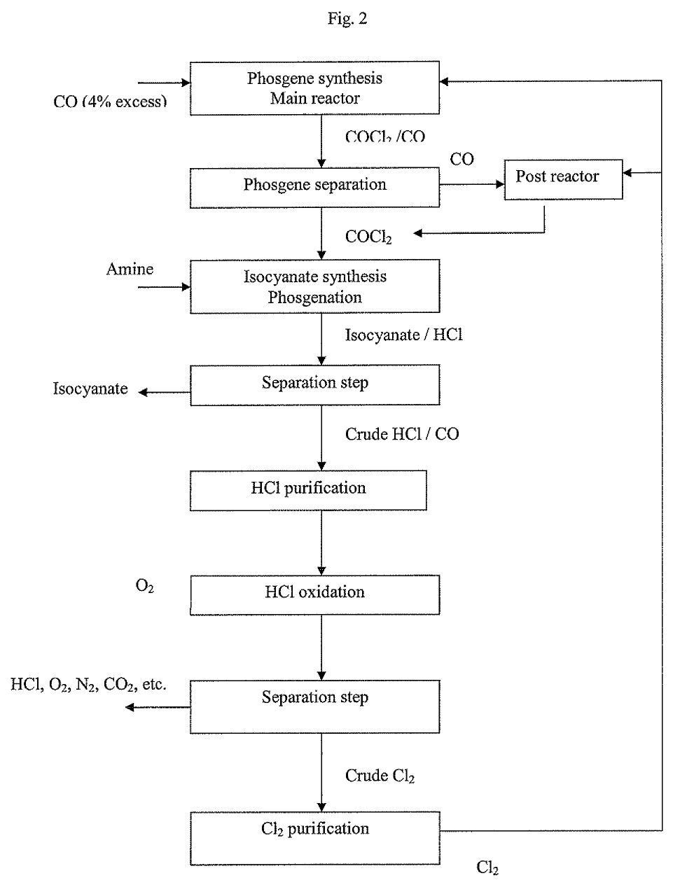

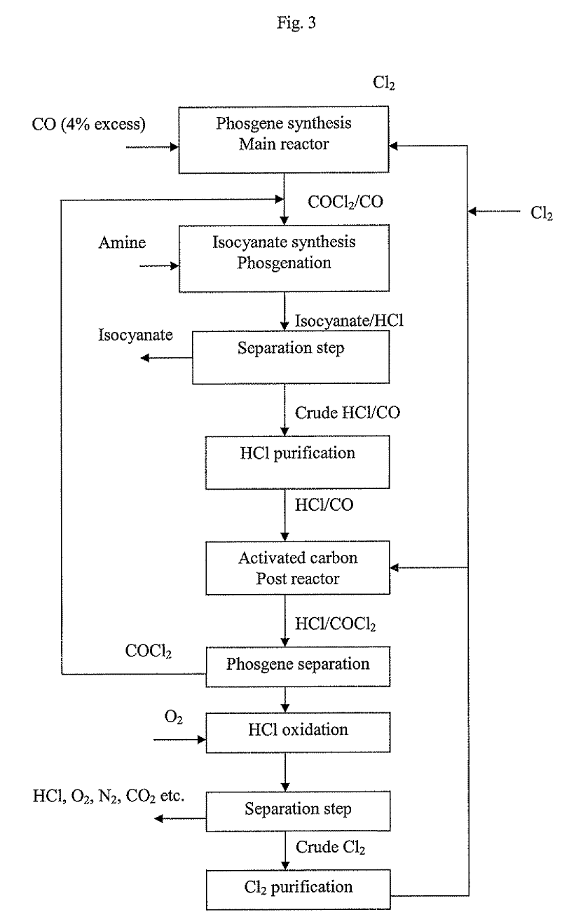

FIGS. 1 and 3 described below illustrate processes performed according to two embodiments of the invention. By contrast, FIG. 2 illustrates a conventional process wherein the CO formed in the phosgene synthesis is first separated off by condensation of the phosgene and then reacted with Cl₂ in a post-combiner to form phosgene. The disadvantage of this process, as explained above, lies in the fact that the condensation of the phosgene is very energy intensive.

FIG. 1 depicts, as a flow chart, a portion of a process according to one embodiment of the invention, wherein an HCl-containing waste gas from an isocyanate synthesis is treated to remove the carbon monoxide present The HCl/CO waste gas which originated from an isocyanate production process, is first reacted, preferably on an activated carbon catalyst, using chlorine to form an HCl/phosgene gas mixture. This is followed by the separation of the phosgene, which can preferably be fed back into the phosgenation or isocyanate production process. After separation of the phosgene, the remaining HCl gas is subjected to HCl oxidation (e.g., a Deacon process) to produce chlorine. After separation of the chlorine produced, it is further possible to feed the resulting gases (e.g., HCl, O₂, N₂, CO₂, etc.) back into the HCl oxidation stage (not shown).

FIG. 3 depicts, as a flow chart, a process according to one embodiment of the invention. In the embodiment shown, the carbon monoxide which is used in excess in the phosgene synthesis does not need to be separated off, eliminating the need for an energy-intensive condensation of the phosgene, and can be fed to the isocyanate synthesis as a COCl₂/CO mixture. There is also no need for a post-reactor. The CO-containing phosgene is therefore used in the isocyanate synthesis (or other synthesis) as it is. After separating off the isocyanate that has formed (the “Separation step” in FIG. 3), the resulting CO/HCl-containing waste gas, which can first be subjected to HCl purification, is subjected to a carbon monoxide separation according to the invention to form phosgene, which can be separated off and fed back into the phosgenation reaction. The HCl gas depleted in CO, which preferably contains less than about 0.5 vol. % CO, is then preferably subjected to a Deacon process, i.e., the catalytic oxidation of hydrogen chloride with oxygen to form Cl₂. The Cl₂ formed is separated off and fed back into the phosgene synthesis processes. The residual gas can optionally be fed back into the Deacon process again. The isocyanate synthesis is performed in a manner that is known per se. Phosgene obtained by a process according to the invention can then be used according to the processes known from the prior art for the production of, for example, TDI or MDI from TDA or MDA respectively. The hydrogen chloride forming again during the phosgenation of TDA and MDA can then be reacted to form chlorine using the processes described.

As a result of the process according to the invention, the carbon monoxide content in the HCl stream is clearly reduced, leading to a slowing of the deactivation of the Deacon catalyst in the next step by uncontrolled temperature increase. At the same time, the valuable carbon monoxide can be reused by conversion to phosgene.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A process comprising:
a) reacting carbon monoxide with chlorine to form phosgene;
b) reacting the phosgene with an organic amine to form an organic isocyanate and a waste gas comprising hydrogen chloride and carbon monoxide;
c) reacting the carbon monoxide in the waste gas with chlorine to form an intermediate gas comprising additional phosgene; and
d) separating the additional phosgene from the intermediate gas.

2. The process according to claim 1, further comprising supplying the additional phosgene to an isocyanate synthesis.

3. The process according to claim 1, further comprising subjecting the intermediate gas to HCl oxidation.

4. The process according to claim 2, further comprising subjecting the intermediate gas to HCl oxidation.

5. The process according to claim 1, wherein carbon monoxide is present in the waste gas in an amount of 0.5 to 15 vol. %.

6. The process according to claim 3, wherein carbon monoxide is present in the waste gas in an amount of 0.5 to 15 vol. %.

7. The process according to claim 1, wherein hydrogen chloride is present in the waste gas in an amount of 20 to 99.5 vol. %.

8. The process according to claim 1, wherein the reaction of the carbon monoxide in the waste gas with chlorine to form additional phosgene is carried out in the presence of a catalyst.

9. The process according to claim 8, wherein the catalyst comprises activated carbon.

10. The process according to claim 1, wherein the reaction of the carbon monoxide in the waste gas with chlorine to form additional phosgene is carried out in a fixed bed reactor on an activated carbon catalyst.

11. The process according to claim 1, wherein the separation of the additional phosgene comprises at least one operation selected from the group consisting of liquefaction of the phosgene, condensation of the phosgene, distillation of the phosgene, rectification of the phosgene, scrubbing of the phosgene with a solvent, and combinations thereof.

12. The process according to claim 1, further comprising supplying the additional phosgene to a phosgenation reaction.

13. The process according to claim 12, wherein the additional phosgene is supplied to the reaction with the organic amine to form the organic isocyanate and waste gas.
